(19) United States
(12) Patent Application Publication
(10) Pub. No.: US 2024/0000093 A1
(43) Pub. Date: Jan. 4, 2024

(54) CULTURED MILK PRODUCT COMPOSITIONS

(71) Applicant: BIOMILQ, Inc., Durham, NC (US)

(72) Inventor: Leila Strickland, Hillsborough, NC (US)

(21) Appl. No.: 18/255,571

(22) PCT Filed: Nov. 30, 2021
(86) PCT No.: PCT/US2021/061147

§ 371 Date: Jun. 1, 2023

Related U.S. Application Data
(60) Provisional application No. 63/120,378, filed on Dec. 2, 2020.

(51) Int. Cl. A23C 23/00; C12N 5/071; A23J 3/10; B65D 85/80; B65D 81/20

(52) U.S. Cl. CPC A23C 23/00; C12N 5/0631; B65D 81/2007; B65D 85/80; A23J 3/10

(57) ABSTRACT

Disclosed herein are cultured milk product compositions that comprise protein, lipid, and oligosaccharide components at concentrations that mimic and/or are substantially similar to bovine milk. The cultured milk product compositions include one or more milk components produced in vitro and/or ex vivo from cultured bovine mammary cells.

### CROSS-REFERENCE

This application claims the benefit of priority from U.S. Provisional Application No. 63/120,378, filed on Dec. 2, 2020, the contents of which are incorporated herein by reference in its entirety.

### FIELD

This disclosure relates to cultured milk product compositions that comprise protein, lipid, and oligosaccharide components and component concentrations that mimic bovine milk, which cultured milk product compositions are produced in vitro and/or ex vivo from cultured bovine mammary cells.

### BACKGROUND

Milk is a complex suspension of nutrients, including fats, sugars, proteins, vitamins, and minerals. Humans have consumed the milks of other species since prehistoric times, and dairy remains a staple of the human diet. Bovine species are the most commonly used in dairy production, based on their high productivity and their widespread availability and adaptability as a species. However, dairy production is an agriculturally intensive process with substantial environmental impacts, including contributions to greenhouse gas production, as well as detrimental effects on land and water resources. Therefore, there is a need for improved cultured milk products that reduce or eliminate the environmental impact of conventional, bovine-based milk production.

### SUMMARY OF THE DISCLOSURE

Disclosed herein, in certain embodiments, are cultured milk product compositions that comprise protein, lipid, and oligosaccharide components and component concentrations that mimic and/or are substantially similar to bovine milk and are produced in vitro and/or ex vivo from cultured bovine mammary cells.

Disclosed herein, in certain embodiments, is a cultured milk product comprising specified concentrations or amounts (e.g., in grams per liter (g/L)) of protein components, lipid components, bovine milk oligosaccharides (BMOs), and lactose, wherein at least one of the protein components, lipid components, BMOs, and lactose is produced by cultured bovine mammary epithelial cells.

In some embodiments of the cultured milk product, the protein component comprises about 21-50 g/L of the cultured milk product, and in some embodiments, the protein component can comprise one or more of whey protein and casein protein. Casein protein, in some embodiments, can comprise one or more of beta-casein, kappa-casein, and alpha-casein, and wherein, in some embodiments, the alpha-casein can comprise one or more of alphaₛ₁-casein and alphaₛ₂-casein. In some embodiments, one or more of the protein components comprise specified concentrations.

In some embodiments, the protein component further comprises one or more of beta-lactoglobulin, alpha-lactalbumin, lysozyme, lactoferrin, and serum albumin that, in some embodiments, have specified concentrations.

In some embodiments, one or more of the protein components are of bovine origin.

In some embodiments of the cultured milk product, the lipid component comprises about 35-55 g/L of the cultured milk product, and in some embodiments of the cultured milk product, the lipid component can comprise one or more of triacylglycerides, diacylglycerides, saturated fatty acids, monounsaturated fatty acids, polyunsaturated fatty acids, cholesterol, and phospholipids. In some embodiments, the triacylglycerides comprise specified concentrations. In some embodiments, diacylglycerides comprise specified concentrations. In some embodiments, saturated fatty acids comprise a specified concentration, and in some embodiments, the saturated fatty acid component can comprise one or more of myristic acid, palmitic acid, and lauric acid. In some embodiments, the one or more of myristic acid, palmitic acid, and lauric acid comprise specified concentrations, and in some embodiments, palmitic acid is enriched in sn-2 configuration.

In some embodiments, monounsaturated fatty acids have specified concentrations. In some embodiments, the monounsaturated fatty acids comprise oleic acid, which, in some embodiments, has a specified concentration.

In some embodiments, polyunsaturated fats have specified concentrations. In some embodiments, the polyunsaturated fats comprise one or more of linoleic acid, conjugated linoleic acid, and alpha-linoleic acid that, in some embodiments, have specified concentrations.

Some embodiments of the cultured milk product comprise cholesterol that, in some embodiments, has specified concentrations.

Some embodiments of the cultured milk product comprise one or more of phospholipids that have specified concentrations.

In some embodiments of the cultured milk product, the milk oligosaccharide comprise about 0.01-0.15 g/L, and in some embodiments, the milk oligosaccharide component can comprise one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL), which, in some embodiments, comprise specified concentrations.

Disclosed herein, in certain embodiments, the cultured milk product comprises about 28-40 grams per liter (g/L) protein components, about 35-55 g/L lipid components, about 0.01-0.15 g/L milk oligosaccharides (MOs), and about 40-60 g/L lactose, wherein the protein components comprise one or more of whey, beta-casein, kappa-casein, alphaₛ₁-casein, alphaₛ₂-casein, beta-lactoglobulin, alpha-lactalbumin, lysozyme, lactoferrin, and serum albumin, wherein the lipid components comprise one or more of triacylglycerides, diacylglycerides, saturated fatty acids, monounsaturated fatty acids, polyunsaturated fatty acids, cholesterol, and phospholipids, wherein the bovine milk oligosaccharide component comprises one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL), wherein saturated fatty acids comprise one or more of myristic acid, palmitic acid, and lauric acid, wherein monounsaturated fatty acids comprise oleic acid, wherein polyunsaturated fatty acids comprise one or more of linoleic acid, conjugated linoleic acid, and alpha-linoleic acid, and wherein at least one of the protein components, lipid components, BMOs, and lactose is produced by cultured bovine mammary epithelial cells. In some embodiments, one or more of the specific protein, lipid, and milk oligosaccharide components all have specified concentrations.

Some embodiments of the cultured milk product are isolated from cultured bovine mammary epithelial cells (i.e., isolated from the secretion that is produced by cultured bovine mammary epithelial cells and/or the cell culture supernatant), which, in some embodiments, comprise one or more immortalized bovine mammary cell lines. In some embodiments, the cultured bovine mammary epithelial cells are derived from one or more primary bovine mammary tissue samples, which, in some embodiment, derive from needle aspiration, surgical explant of bovine mammary gland tissue or other type of tissue removal method. In some embodiments, the cultured bovine mammary epithelial cells are isolated from raw bovine milk or descend from one or more bovine mammary epithelial cells that were originally isolated from raw bovine milk. In some embodiments, the one or more primary bovine mammary tissue samples can comprise tissue or cells collected from bovine mammary parenchyma.

In some embodiments, the primary bovine mammary tissue further comprises one or more myoepithelial cells and/or comprises one or more stem cells.

In some embodiments, the cultured milk product is sterile, and in some embodiments, the cultured milk product is sterile without pasteurization. In some embodiments, the milk product is free of immunoglobulin protein. In some embodiments, the cultured milk product comprises at least about 80% of the overall macromolecular composition of bovine milk, or at least about 85% of the overall macromolecular composition of bovine milk. In some embodiments, the cultured milk product comprises at least about 90% of the overall macromolecular composition of bovine milk. In some embodiments, the cultured milk product comprises at least about 95% of the overall macromolecular composition of bovine milk. In some embodiments, the cultured milk product comprises at least about 97% of the overall macromolecular composition of bovine milk. In some embodiments, the cultured milk product comprises at least about 98% of the overall macromolecular composition of bovine milk. In some embodiments, the cultured milk product comprises at least about 99% of the overall macromolecular composition of bovine milk. In some embodiments, non-protein nitrogen content comprises at least about 10% of total nitrogen content. In some embodiments, non-protein nitrogen content comprises at least about 15% of total nitrogen content. In some embodiments, non-protein nitrogen content comprises at least about 20% of total nitrogen content. In some embodiments, non-protein nitrogen content comprises at least about 25% of total nitrogen content. In some embodiments, non-protein nitrogen content comprises at least about 30% of total nitrogen content.

Disclosed herein, in certain embodiments, is a frozen cultured milk product, a lyophilized cultured milk product, a filtered cultured milk product, an extracted cultured milk product, and a containerized cultured milk product comprising the cultured milk product of the disclosure.

In some embodiments, the cultured milk product can comprise between about 450-900 kcal/L available energy content, and in some embodiments, between about 40-60% of the available energy content is from lipid components in the cultured milk product.

In some embodiments, the cultured milk product comprises a specified amount of macromolecular content. In some embodiments of the cultured milk product that contain casein protein, the casein protein can take the form of a micelle having specified characteristics. In some embodiments, the cultured milk product comprises milk fat globules in specified concentrations and in specified forms.

In some embodiments of the cultured milk product that contain triacylglycerides, specified amounts of the triglycerides can comprise palmitic acid at the sn-2 position, and in some embodiments, omega-6 to omega-3 ratios are specified.

Having described the present disclosure, the same will be explained in greater detail in the following examples, which are included herein for illustration purposes only, and which are not intended to be limiting to the disclosure.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Numerous variations and additions to the various embodiments suggested herein will be apparent to those skilled in the art in light of the instant disclosure, which do not depart from the instant disclosure. Hence, the following specification is intended to illustrate some particular embodiments of the disclosure, and not to exhaustively specify all permutations, combinations, and variations thereof.

Unless the context indicates otherwise, it is specifically intended that the various features described herein can be used in any combination. Moreover, in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this disclosure belongs. The terminology used in the description herein is for the purpose of describing particular embodiments only and is not intended to be limiting.

All publications, patent applications, patents, nucleotide sequences, amino acid sequences and other references mentioned herein are incorporated by reference in their entireties for all purposes.

### Definitions

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, elements, and/or components, but do not preclude the presence or addition of one or more other features, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Moreover, the present disclosure also contemplates that in some embodiments, any feature or combination of features set forth herein can be excluded or omitted.

Furthermore, the term “about,” as used herein when referring to a measurable value such as an amount of a compound or agent of this disclosure, dose, time, temperature, and the like, is meant to encompass variations of 10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the disclosure. Thus, the term “consisting essentially of” as used herein should not be interpreted as equivalent to “comprising.”

As used herein, the term “polypeptide” encompasses both peptides and proteins, and does not require any particular amino acid length or tertiary structure unless indicated otherwise.

The term “polarized” as used herein in reference to cells and/or monolayers of cells refers to a spatial status of the cell wherein there are two distinct surfaces of the cell, e.g., an apical surface and a basal surface, which may be different. In some embodiments, the distinct surfaces of a polarized cell comprises different surface and/or transmembrane receptors and/or other structures. In some embodiments, individual polarized cells in a continuous monolayer have similarly-oriented apical surfaces and basal surfaces. In some embodiments, individual polarized cells in a continuous monolayer have communicative structures between individual cells (e.g., tight junctions) to allow cross communication between individual cells and to create separation (e.g., compartmentalization) of the apical compartment and basal compartment.

As used herein, “apical surface” means the surface of a cell that faces an external environment or toward a cavity, for example the cavity of an internal organ. With respect to mammary epithelial cells, the apical surface is the surface from which the cultured milk product is excreted.

As used herein, “basal surface” means the surface of a cell that is in contact with a surface, e.g., the extracellular matrix of a bioreactor.

As used herein, “bioreactor” means a device or system that supports a biologically active environment that enables the production of a culture milk product described herein from mammary cells described herein.

The term “lactogenic” as used herein refers to the ability to stimulate production and/or secretion of milk. A gene or protein (e.g., prolactin) may be lactogenic, as may any other natural and/or synthetic product. In some embodiments, a lactogenic culture medium comprises prolactin, thereby stimulating production of milk by cells in contact with the culture medium.

As used herein, the compositions described in the present disclosure are referred to interchangeably as (the singular or plural forms of) “nutritional compositions substantially similar to bovine milk,” “cultured milk products,” “cultured milk compositions,” “cultured milk product compositions” or equivalent as made clear by the context.

As used herein, by “isolate” (or grammatical equivalents, e.g., “extract”) a product, it is meant that the product is at least partially separated from at least some of the other components in the starting material.

As used herein, the term “food grade” refers to materials considered non-toxic and safe for consumption (e.g., human and/or other animal consumption), e.g., as regulated by standards set by the U.S. Food and Drug Administration.

### Cultured Bovine Milk

Bovine milk and bovine milk products are nutritious food items containing numerous essential nutrients such as, oleic acid, conjugated linoleic acid, omega-3 fatty acids, vitamins, minerals and bioactive compounds such as antioxidants. However, due to the extreme processes that bovine milk goes through and the exposure of cows to antibiotics, hormones, genetic selection, change in diet, and genetically-modified substances, there are concerns associated with drinking milk from cows. Cows release contaminants and toxins through their milk, as milk is a natural exit-portal for substances that the body cannot use. Examples of potential contaminants of bovine milk include hormones (e.g., pituitary, steroid, hypothalamic, and thyroid hormones), gastrointestinal peptides (e.g., nerve and epidermal growth factors, and the growth inhibitors MDGI and MAF), rBGH or recombinant bovine growth hormone (a genetically engineered hormone injected into cows to increase milk production which has been linked to breast, colon and prostate cancer), pus from infected bovine udders, and/or antibiotics or pharmaceuticals which have been administered to cows.

Bovine milk also harbors a complex microbial community, including microorganisms that are of concern from a food quality or safety perspective. The bovine milk microbiota is the focus of constant attention and testing. Such testing occurs daily on both raw and pasteurized bovine milk. The microbial composition of milk is influenced by several different parameters such as, in the case of raw bovine milk, the microorganisms present in the teat canal, on the surface of teat skin, in the surrounding air, in feed, as well as other environmental factors including bovine housing conditions, the quality of the water supply, and equipment hygiene. The microbiota of pasteurized bovine milk is thought to be determined by the percentage of thermoduric bacteria that survive pasteurization temperatures and by the bacteria associated with post-pasteurization contamination. It has been suggested that the potential for microbes, usually considered to be eliminated by pasteurization, to survive commercial pasteurization and the apparent presence of these populations in commercial milk, and there is a potential effect on milk quality, shelf-life, and milk-based products.

The present disclosure relates to cultured milk product compositions that comprise protein, lipid, and oligosaccharide components and component concentrations that mimic bovine milk, which compositions are produced, at least in part, by in vitro and/or ex vivo cultured bovine mammary cells.

Contemplated cultured milk product compositions of the present disclosure can be defined by total levels of protein, lipid, and carbohydrate (Tables 1A-1C) and/or by a signature of specific macronutrient components (Tables 2A-2C) present in concentrations and proportions consistent with bovine milk.

TABLE 1A
Macromolecular Composition of Functional Nutrition Products
Collected from Bovine Mammary Epithelial Cells.

| Macromolecular Fraction | Concentration, g/L |
|---|---|
| Protein | 14-60 |
| Lipid | 18-83 |
| Milk oligosaccharides | 0.23 |
| Lactose | 20-90 |
| Total macromolecular content | 50-235 |
| Energy (kcal/L) | 225-1350 |

Long-chain fatty acids linoleic acid and alpha-linoleic acid are not synthesized by mammalian cells and are supplemented in cell culture media.

TABLE 1B
Macromolecular Composition of Functional Nutrition Products
Collected from Bovine Mammary Epithelial Cells.

| Macromolecular Fraction | Concentration, g/L |
|---|---|
| Protein | 21-50 |
| Lipid | 26-41 |
| Milk oligosaccharides | 0.008-0.19 |
| Lactose | 30-75 |
| Total macromolecular content | 75-170 |
| Energy (kcal/L) | 338-1125 |

Long-chain fatty acids linoleic acid and alpha-linoleic acid are not synthesized by mammalian cells and are supplemented in cell culture media.

TABLE 1C
Macromolecular Composition of Functional Nutrition Products
Collected from Bovine Mammary Epithelial Cells.

| Macromolecular Fraction | Concentration, g/L |
|---|---|
| Protein | 28-40 |
| Lipid | 35-55 |
| Milk oligosaccharides | 0.01-0.15 |
| Lactose | 40-60 |
| Total macromolecular content | 100-160 |
| Energy (kcal/L) | 450-900 |

Long-chain fatty acids linoleic acid and alpha-linoleic acid are not synthesized by mammalian cells and are supplemented in cell culture media.

In some embodiments, the concentrations of components indicated in Tables 1A-1C can vary, each individually, for example, by having a concentration that is greater than that indicated by 0.1 fold, or 0.2 fold, or 0.3 fold, or 0.4 fold, or 0.5 fold, or 0.6 fold, or 0.7 fold, or 0.8 fold, or 0.9 fold, or 1, 2, 3, 4, 5, 6, 7, 8, 9, or 10 fold.

In some embodiments, the concentrations of components indicated in Tables 1A-1C can vary, each individually, for example, by having a concentration that is less than that indicated by 0.1 fold, or 0.2 fold, or 0.3 fold, or 0.4 fold, or 0.5 fold, or 0.6 fold, or 0.7 fold, or 0.8 fold, or 0.9 fold.

In some embodiments, cultured milk products are contemplated herein that include a subset of the components (i.e., macromolecular fractions) of Tables 1A-1C. In other embodiments, cultured milk products contemplated herein can exclude one or more of the components (i.e., macromolecular fractions) of Tables 1A-1C.

TABLE 2A
Macromolecular Content and Concentration Ranges of Functional
Nutrition Products Derived from Bovine Mammary Epithelial.

| Macromolecular Fraction | Concentration, g/L |
|---|---|
| Protein¹⁻³ | |
| Total Protein | 14-60 |
| Total Casein | 8-53 |
| Total Whey Proteins | 1-24 |
| β-Casein | 3.5-18 |
| α_{S1}-Casein | 3.5-18 |
| α_{S2}-Casein | 1-6 |
| κ-Casein | 0.5-6 |
| β-Lactoglobulin | 1-8 |
| α-Lactalbumin | 0.25-3 |
| Lysozyme (μg/L) | 2.5-23 |
| | (μg/L) |
| Lactoferrin | 0.005-0.8 |
| Serum albumin | 0.03-3 |
| Lipids² | |
| Total lipids | 18-83 |
| Triacylglycerides | 15-80 |
| Diacylglycerides | 0.15-3 |
| Cholesterol | 0.1-6 |
| Phospholipids | 0.05-2 |
| Free fatty acids | 0.05-2 |
| Fatty acids² | |
| Saturated fatty acids | 8-38 |
| Myristic acid (C14:0) | 0.5-6 |
| Palmitic acid (C16:0) | 3-15 |
| Lauric acid (C12:0) | 0.3-2 |
| Monounsaturated fatty acids | 2.5-18 |
| Oleic acid (C18:1 n-9 Z) | 3-15 |
| Polyunsaturated fatty acids | |
| Linoleic acid, LA | 0.25-3 |
| (C18:2 n-6 Z) | |
| Conjugated linoleic acid, (9c,11t-CLA) | 0.025-0.23 |
| α-Linolenic acid, ALA (C18:3 n-3) | 0.25-2.3 |
| Lactose¹ | 20-90 |
| Oligosaccharides^{4, 5} | |
| Total Oligosaccharides | 0.005-0.23 |
| 6′-Sialyllactose (6′-SL) | 0.005-0.15 |
| 6′-sialyl-n-acetyllactosamine (6′-SLN) | 0.003-0.03 |
| Disialyllactose (DSL) | <0.005 |
| Galactosaminuyllactose (GNL) | 0.001-0.009 |
| 3′-Sialyllactose (3′-SL) | 0.013-0.23 |

TABLE 2B
Macromolecular Content and Concentration Ranges of Functional
Nutrition Products Derived from Bovine Mammary Epithelial Cells.

| Macromolecular Fraction | Concentration, g/L |
|---|---|
| Protein¹⁻³ | |
| Total Protein | 21-50 |
| Total Casein | 12-44 |
| Total Whey Proteins | 1.5-20 |
| β-Casein | 5.3-15 |
| α_{S1}-Casein | 5.3-15 |
| α_{S2}-Casein | 1.5-5 |
| κ-Casein | 0.8-5 |
| β-Lactoglobulin | 1.5-6.3 |
| α-Lactalbumin | 0.38-2.5 |
| Lysozyme (μg/L) | 3.8-19 |
| | (μg/L) |
| Lactoferrin | 0.008-0.63 |
| Serum albumin | 0.038-2.5 |
| Lipids² | |
| Total lipids | 26-69 |
| Triacylglycerides | 23-68 |
| Diacylglycerides | 0.23-2.5 |
| Cholesterol | 0.15-5 |
| Phospholipids | 0.08-1.3 |
| Free fatty acids | 0.08-1.3 |
| Fatty acids² | |
| Saturated fatty acids | 11.3-31.3 |
| Myristic acid (C14:0) | 0.75-5 |
| Palmitic acid (C16:0) | 4.5-12.5 |
| Lauric acid (C12:0) | 0.45-1.25 |
| Monounsaturated fatty acids | 3.8-15 |
| Oleic acid (C18:1 n-9 Z) | 4.5-12.5 |
| Polyunsaturated fatty acids | |
| Linoleic acid, LA | 0.38-2.5 |
| (C18:2 n-6 Z) | |
| Conjugated linoleic acid, (9c, 11t-CLA) | 0.038-0.19 |
| α-Linolenic acid, ALA (C18:3 n-3) | 0.38-1.9 |
| Lactose¹ | 30-75 |
| Oligosaccharides^{4, 5} | |
| Total Oligosaccharides | 0.0075-0.19 |
| 6′-Sialyllactose (6′-SL) | 0.0075-0.13 |
| 6′-sialyl-n-acetyllactosamine (6′-SLN) | 0.0038-0.025 |
| Disialyllactose (DSL) | <0.008 |
| Galactosaminuyllactose (GNL) | 0.0015-0.0075 |
| 3′-Sialyllactose (3′-SL) | 0.019-0.19 |

TABLE 2C
Macromolecular Content and Concentration Ranges of Functional
Nutrition Products Derived from Bovine Mammary Epithelial Cells.

| Macromolecular Fraction | Concentration, g/L |
|---|---|
| Protein¹⁻³ | |
| Total Protein | 28-40 |
| Total Casein | 16-35 |
| Total Whey Proteins | 2-16 |
| β-Casein | 7-12 |
| α_{S1}-Casein | 7-12 |
| α_{S2}-Casein | 2-4 |
| κ-Casein | 1-4 |
| β-Lactoglobulin | 2-5 |
| α-Lactalbumin | 0.5-2 |
| Lysozyme (μg/L) | 5-15 |
| | (μg/L) |
| Lactoferrin | 0.01-0.5 |
| Serum albumin | 0.05-2 |
| Lipids² | |
| Total lipids | 35-55 |
| Triacylglycerides | 30-54 |
| Diacylglycerides | 0.3-2 |
| Cholesterol | 0.2-4 |
| Phospholipids | 0.1-1 |
| Free fatty acids | 0.1-1 |
| Fatty acids² | |
| Saturated fatty acids | 15-25 |
| Myristic acid (C14:0) | 1-4 |
| Palmitic acid (C16:0) | 6-10 |
| Lauric acid (C12:0) | 0.6-1 |
| Monounsaturated fatty acids | 5-12 |
| Oleic acid (C18:1 n-9 Z) | 6-10 |
| Polyunsaturated fatty acids | 0.5-10 |
| Linoleic acid, LA | 0.5-2 |
| (C18:2 n-6 Z) | |
| Conjugated linoleic acid, (9c, 11t-CLA) | 0.05-0.15 |
| α-Linolenic acid, ALA (C18:3 n-3) | 0.5-1.5 |
| Lactose¹ | 40-60 |
| Oligosaccharides^{4, 5} | |
| Total Oligosaccharides | 0.01-0.15 |
| 6′-Sialyllactose (6′-SL) | 0.01-0.1 |
| 6′-sialyl-n-acetyllactosamine (6′-SLN) | 0.005-0.02 |
| Disialyllactose (DSL) | <0.01 |
| Galactosaminuyllactose (GNL) | 0.002-0.006 |
| 3′-Sialyllactose (3′-SL) | 0.025-0.15 |

In some embodiments, the concentrations of components indicated in Tables 2A-C can vary, each individually, for example, by having a concentration that is greater than that indicated by 0.1 fold, or 0.2 fold, or 0.3 fold, or 0.4 fold, or 0.5 fold, or 0.6 fold, or 0.7 fold, or 0.8 fold, or 0.9 fold, or 1, 2, 3, 4, 5, 6, 7, 8, 9, or 10 fold.

In some embodiments, the concentrations of components indicated in Tables 2A-C can vary, each individually, for example, by having a concentration that is less than that indicated by 0.1 fold, or 0.2 fold, or 0.3 fold, or 0.4 fold, or 0.5 fold, or 0.6 fold, or 0.7 fold, or 0.8 fold, or 0.9 fold.

In some embodiments, cultured milk products are contemplated herein that include a subset of the components (i.e., macromolecular fractions) of Tables 2A-C. In other embodiments, cultured milk products contemplated herein can exclude one or more of the components (i.e., macromolecular fractions) of Tables 2A-C.

Disclosed herein, in certain embodiments, are a cultured milk products comprising about 28-40 grams per liter (g/L) protein components, about 35-55 g/L lipid components, about 0.01-0.15 g/L bovine milk oligosaccharides (BMOs), and about 40-60 g/L lactose, wherein at least one of the protein components, lipid components, MOs, and lactose is produced by cultured bovine mammary epithelial cells.

In some embodiments of the cultured milk product, the protein component can comprise whey protein, and in some embodiments the whey protein can have a concentration of about 1-24 g/L in the cultured milk product. In some embodiments, the protein component can comprise casein protein, and in some embodiments, casein protein can comprise one or more of beta-casein, kappa-casein, and alpha-casein. In some embodiments, the beta-casein can have a concentration of about 7-12 g/L, the kappa-casein can have a concentration of about 1-4 g/L, and the alpha-casein can have a concentration of about 9-16 g/L in the cultured milk product. In some embodiments, the alpha-casein can comprise one or more of alphaₛ₁-casein and alphaₛ₂-casein, and in some embodiments, the alphaₛ₁-casein is at least 1.5 fold, or about 2 fold, or about 2.5 fold, or about 3 fold, or about 3.5 fold, or about 4 fold more abundant than alphaₛ₂-casein. In some embodiments, the alphaₛ₁-casein can have a concentration of about 7-12 g/L in the cultured milk product, and in some embodiments, the alphaₛ₂-casein can have a concentration of about 2-4 g/L in the cultured milk product. In some embodiments, the beta-casein can comprise greater than about 50% of total casein content.

In some embodiments of the cultured milk product, the protein component can further comprise one or more of beta-lactoglobulin, alpha-lactalbumin, lysozyme, lactoferrin and serum albumin, and in some embodiments, the beta-lactoglobulin can have a concentration of about 2-5 g/L in the cultured milk product and/or the alpha-lactalbumin can have a concentration of about 0.5-2 g/L in the cultured milk product and/or the lysozyme can have a concentration of about 5-15 μg/L in the milk product and/or the lactoferrin can have a concentration of about 0.01-0.5 g/L in the cultured milk product and/or the serum albumin can have a concentration of about 0.05-2 g/L in the cultured milk product.

In some embodiments, the protein components are of bovine origin.

In some embodiments of the cultured milk product, the lipid component can comprise one or more of triacylglycerides, diacylglycerides, saturated fatty acids, monounsaturated fatty acids, polyunsaturated fatty acids, cholesterol, and phospholipids. In some embodiments, triacylglycerides can have a concentration of about 30-54 g/L in the cultured milk product, and in some embodiments, diacylglycerides can have a concentration of about 0.33-2 g/L in the cultured milk product. In some embodiments, saturated fatty acids can have a concentration of about 15-25 g/L in the cultured milk product, and in some embodiments, the saturated fatty acid component can comprise one or more of myristic acid, palmitic acid, and lauric acid, where, in some embodiments, myristic acid can have a concentration of about 1-4 g/L in the cultured milk product, and, in some embodiments, palmitic acid can have a concentration of about 6-10 g/L in the cultured milk product, and, in some embodiments, lauric acid can have a concentration of about 0.6-1 g/L in the cultured milk product.

In some embodiments of the cultured milk product, monounsaturated fatty acids can have a concentration of about 5-12 g/L in the cultured milk product, and, in some embodiments, monounsaturated fatty acid can comprise oleic acid, which oleic acid, in some embodiments, can have a concentration of about 6-10 g/L in the cultured milk product.

In some embodiments of the cultured milk product, polyunsaturated fats can have a concentration of about 0.5-10 g/L in the cultured milk product, and, in some embodiments, the polyunsaturated fats can comprise one or more of linoleic acid, conjugated linoleic acid, and alpha-linoleic acid. In some embodiments, linoleic acid can have a concentration of about 0.5-2 g/L in the cultured milk product, and in some embodiments, conjugated linolenic acid can have a concentration of about 0.05-0.15 g/L in the cultured milk product, and in some embodiments, alpha-linoleic acid can have a concentration of about 0.5-1.5 g/L in the cultured milk product.

In some embodiments, linoleic acid has a concentration of about 0.5-2 g/L, conjugated linolenic acid has a concentration of about 0.05-0.15 g/L, and alpha-linoleic acid has a concentration of about 0.5-1.5 g/L in the cultured milk product.

In some embodiments, the cultured milk product can comprise cholesterol, which, in some embodiments can have a concentration of about 0.2-4 g/L in the cultured milk product.

In some embodiments, the cultured milk product can comprise phospholipids, which, in some embodiments, can have a concentration of about 0.1-1 g/L in the cultured milk product.

In some embodiments of the cultured milk product, the milk oligosaccharide component can comprise one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL).

In some embodiments, the one or more milk oligosaccharides comprises 6′-Sialyllactose (6′-SL), which, in some embodiments, can have a concentration of about 0.01-0.1 g/L in the cultured milk product.

In some embodiments, the one or more oligosaccharides comprises 6′-sialyl-n-acetyllactosamine (6′-SLN), which, in some embodiments, can have a concentration of about 0.005-0.02 g/L in the cultured milk product. In some embodiments, the one or more oligosaccharides comprises Disialyllactose (DSL), which, in some embodiments, can have a concentration of less than about 0.01 g/L in the cultured milk product. In some embodiments, the one or more oligosaccharides comprises Galactosaminuyllactose (GNL), which, in some embodiments, can have a concentration of about 0.002-0.006 g/L in the cultured milk product. In some embodiments, the one or more oligosaccharides comprises 3′-Sialyllactose (3′-SL), which, in some embodiments, can have a concentration of about 0.025-0.15 g/L in the cultured milk product. In some embodiments, the cultured milk product can comprise about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL), and about 0.025-0.15 g/L 3′-SL (3′-sialyllactose).

In another aspect of the disclosure, the cultured milk product comprises about 28-40 grams per liter (g/L) protein components, about 35-55 g/L lipid components, about 0.01-0.15 g/L milk oligosaccharides (MOs), and about 40-60 g/L lactose, wherein the protein components comprise one or more of whey, beta-casein, kappa-casein, alphaₛ₁-casein, alphaₛ₂-casein, beta-lactoglobulin, alpha-lactalbumin, lysozyme, lactoferrin, and serum albumin, wherein the lipid components comprises one or more of triacylglycerides, diacylglycerides, saturated fatty acids, monounsaturated fatty acids, polyunsaturated fatty acids, cholesterol, and phospholipids, wherein the bovine milk oligosaccharide component comprise one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL), wherein saturated fatty acids comprise one or more of myristic acid, palmitic acid, and lauric acid, wherein monounsaturated fatty acids comprise oleic acid, wherein polyunsaturated fatty acids comprise one or more of linoleic acid, conjugated linoleic acid, and alpha-linoleic acid, and wherein at least one of the protein components, lipid components, BMOs, and lactose is produced by cultured bovine mammary epithelial cells.

In some embodiments, the cultured milk product comprises about 2-16 g/L whey, about 7-12 g/L beta-casein, about 1-4 g/L kappa-casein, about 7-12 g/L alphaₛ₁-casein, about 2-4 g/L alphaₛ₂-casein, about 2-5 g/L beta-lactoglobulin, about 0.5-2 g/L alpha-lactalbumin, about 5-15 μg/L lysozyme, about 0.01-0.5 g/L lactoferrin, about 0.05-2 g/L serum albumin, about 30-54 g/L triacylglycerides, about 0.3-2 g/L diacylglycerides, about 15-25 g/L saturated fatty acids, about 5-12 g/L monounsaturated fatty acids, about 0.5-10 g/L polyunsaturated fatty acids, about 0.2-4 g/L cholesterol, about 0.1-1 g/L phospholipids, about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL) and about 0.025-0.15 g/L 3′-Sialyllactose (3′-SL), wherein the saturated fatty acids comprise about 1-4 g/L myristic acid, about 6-10 g/L palmitic acid, and about 0.6-1 g/L lauric acid, wherein the monounsaturated fatty acids comprises about 6-10 g/L oleic acid, and wherein the polyunsaturated fatty acids comprise about 0.5-2 g/L linoleic acid, about 0.05-0.15 g/L conjugated linoleic acid, and about 0.5-1.5 g/L alpha-linoleic acid.

In some embodiments of the cultured milk product, the protein components, lipid components, BMOs, and lactose are isolated from cultured mammary epithelial cells (i.e., isolated from the secretion that is produced by cultured bovine mammary epithelial cells and/or the cell culture supernatant), and in some embodiments the cultured bovine mammary epithelial cells can comprise one or more immortalized bovine mammary cell lines. In some embodiments, the cultured bovine mammary epithelial cells are derived from one or more primary bovine mammary tissue samples, and in some embodiments, the one or more primary bovine mammary tissue samples can be derived from a surgical explant of bovine mammary gland tissue and/or from a needle aspiration of bovine mammary gland tissue. In some embodiments, the one or more primary bovine mammary tissue samples can comprise tissue or cells collected from bovine mammary parenchyma. In some embodiments, the cultured bovine mammary epithelial cells are isolated from raw bovine milk or descend from one or more mammary epithelial cells that were originally isolated from raw bovine milk.

In some embodiments, the primary bovine mammary tissue can further comprise one or more myoepithelial cells and/or one or more stem cells. In some embodiments, the cultured bovine mammary epithelial cells can be co-cultured with one or more myoepithelial cell lines and/or with one or more stem cell lines.

In some embodiments, the cultured milk product is sterile, and in some embodiments, the cultured milk product is sterile without pasteurization.

In some embodiments, the cultured milk product is free of immunoglobulin protein.

In some embodiments, the cultured milk product comprises at least about 80% of the overall macromolecular composition of bovine milk. In some embodiments, the cultured milk product comprises at least about 85% of the overall macromolecular composition of bovine milk. In some embodiments, the cultured milk product comprises at least about 90% of the overall macromolecular composition of bovine milk. In some embodiments, the cultured milk product comprises at least about 95% of the overall macromolecular composition of bovine milk. In some embodiments, the cultured milk product comprises at least about 97% of the overall macromolecular composition of bovine milk. In some embodiments, the cultured milk product comprises at least about 98% of the overall macromolecular composition of bovine milk. In some embodiments, the cultured milk product comprises at least about 99% of the overall macromolecular composition of bovine milk. In some embodiments, non-protein nitrogen content comprises at least about 10% of total nitrogen content. In some embodiments, non-protein nitrogen content comprises at least about 15% of total nitrogen content. In some embodiments, non-protein nitrogen content comprises at least about 20% of total nitrogen content. In some embodiments, non-protein nitrogen content comprises at least about 25% of total nitrogen content. In some embodiments, non-protein nitrogen content comprises at least about 30% of total nitrogen content.

In some embodiments, the cultured milk product does not comprise or is substantially free of one or more contaminants and toxins. In some embodiments, the cultured milk product does not comprise or is substantially free of hormones. In some embodiments, the cultured milk product does not comprise or is substantially free of rBGH. In some embodiments, the cultured milk product does not comprise or is substantially free of gastrointestinal peptides. In some embodiments, the cultured milk product does not comprise or is substantially free of antibiotics. In some embodiments, the cultured milk product does not comprise or is substantially free of pharmaceuticals. In some embodiments, the cultured milk product does not comprise or is substantially free of pesticides and/or pesticide residues. In some embodiments, the cultured milk product does not comprise or is substantially free of herbicides and/or herbicide residues. In some embodiments, the cultured milk product does not comprise or is substantially free of mycotoxin. In some embodiments, the cultured milk product does not comprise or is substantially free of perchlorate. In some embodiments, the cultured milk product does not comprise or is substantially free of lead, chromium, tetracycline, enrofloxacin, ciprocin and/or aflatoxin (e.g., aflatoxin M1 (AFM1)).

In some embodiments, the cultured milk product does not comprise or is substantially free of one or more pathogens or microorganisms. In some embodiments, the cultured milk product does not comprise or is substantially free of Brucella, Campylobacter jejuni, Coliforms, Coxiella burnetii, Escherichia coli, Listeria monocytogenes, Mycobacterium bovis and tuberculosis, Mycobacterium paratuberculosis, Psychrotrophic Bacteria, Salmonella spp., and/or Yersinia enterocolitica. In some embodiments, the cultured milk product does not comprise or is substantially free of Proteobacteria, Bacteroidetes, Firmicutes, Actinobacteria, Acidobacteria, Pseudomonas, Brevibacteriaceae, Corynebacteriaceae, Staphylococcaceae, Arthrobacter, Cronobacter, Ruminococcus and/or Faecalibacterium.

In some embodiments, the cultured milk product does not comprise or is substantially free of one or more allergens. In some embodiments, the cultured milk product does not comprise or is substantially free of α-lactalbumin (Bos d 4), β-lactoglobulin (Bos d 5), immunoglobulins (Bos d 7), bovine serum albumin (BSA, Bos d 6), lactoferrin (Bos d lactoferrin), αS1-casein (Bos d 9), αS2-casein (Bos d 10), β-Casein (Bos d 11), and/or κ-Casein (Bos d 12).

In some embodiments, the cultured milk product does not comprise or is substantially free of egg, fish, crustacean shellfish, tree nuts, peanuts, wheat, and soybean allergens. In some embodiments, the cultured milk product does not comprise or is substantially free of egg allergens. In some embodiments, the cultured milk product does not comprise or is substantially free of fish allergens. In some embodiments, the cultured milk product does not comprise or is substantially free of crustacean allergens. In some embodiments, the cultured milk product does not comprise or is substantially free of tree nut allergens. In some embodiments, the cultured milk product does not comprise or is substantially free of peanut allergens. In some embodiments, the cultured milk product does not comprise or is substantially free of wheat allergens. In some embodiments, the cultured milk product does not comprise or is substantially free of soybean allergens.

Disclosed herein, in certain embodiments, is a frozen cultured milk product, comprising the cultured milk product, lyophilized cultured milk product and/or extracted cultured milk product of any embodiment of the disclosure that has been frozen.

Disclosed herein, in certain embodiments, is a lyophilized cultured milk product, comprising the cultured milk product of any embodiment of the disclosure that has been lyophilized.

Disclosed herein, in certain embodiments, is a containerized cultured milk product, comprising one or more of the cultured milk product, the frozen cultured milk product and the lyophilized cultured milk product of any embodiment of the disclosure that is packaged into a container.

In some embodiments of the containerized cultured milk product, the container is sterile, and in some embodiments, the container is vacuum-sealed. In some embodiments, the container is a food grade container, and in some embodiments the container is a canister, a jar, a bottle, a bag, a box, or a pouch.

Disclosed herein, in certain embodiments, is an extracted cultured milk product, comprising one or more components extracted from the cultured milk product of any embodiment of the disclosure. In some embodiments of the extracted cultured milk product, one or more components extracted from the cultured milk product according to any embodiment of the disclosure are lyophilized and/or concentrated to produce a lyophilized and/or concentrated extracted cultured milk product component. In some embodiments, one or more components extracted from the cultured milk product are concentrated by membrane filtration or reverse osmosis.

In some embodiments of the extracted cultured milk product, the one or more extracted components from the collected cultured milk product of any embodiment of the disclosure can comprise one or more of milk protein, lipid, carbohydrate, vitamin, and minerals. In some embodiments of the extracted cultured milk product, one or more of the extracted components of the extracted cultured milk product are packaged in a container.

Disclosed herein, in certain embodiments, is a filtered cultured milk product, comprising one or more components filtered from the cultured milk product of any embodiment of the disclosure. In some embodiments of the filtered cultured milk product, one or more components filtered from the collected cultured milk product are lyophilized or concentrated to produce a lyophilized or a concentrated filtered cultured milk product component. In some embodiments, the one or more components filtered from the collected cultured milk product are concentrated by membrane filtration or reverse osmosis. In some embodiments, the one or more filtered components from the collected cultured milk product comprise milk protein, lipid, carbohydrate, vitamin, and minerals. Filtration can be performed using any filtration process known in the art, e.g., membrane filtration such as microfiltration (MF), ultrafiltration (UF), nanofiltration (NF), and reverse osmosis (RO), vacuum filtration, centrifugal filtration, gravity filtration, multilayer filtration and the like.

In some embodiments, the milk product can comprise between about 450-900 kcal/L available energy content, and in some embodiments, between about 40-60% of the available energy content is from lipid components in the cultured milk product.

In some embodiments, the cultured milk product comprises between about 100 and 160 g/L macromolecular content. In some embodiments of the cultured milk product that contain casein protein, the casein protein can be complexed into one or more micelle structures that have a diameter between about 150 to 250 nm or between about 100 to 200 nm. In some embodiments, the cultured milk product can comprise between about 10⁹ to 10¹¹ milk fat globules per milliliter, wherein the milk fat globules have a core comprising one or more triacylglycerides, the core surrounded by a trilayer comprising one or more phospholipids and one or more membrane proteins. In some embodiments, the milk fat globules have a diameter between about 10 nm and 30 μm, or between about 50 nm and 25 μm, or between about 75 nm and 20 μm, or between about 100 nm and 20 μm, or between about 200 nm and 17.5 μm, or between about 250 nm and 15 μm, or between about 300 nm and 12.5 μm, or between about 400 nm and 10 μm, or between about 500 nm and 7.5 μm, or between about 600 nm and 5 μm, or between about 700 nm and 2.5 μm, or between about 800 nm and 2 μm.

In some embodiments of the cultured milk product that contain triacylglycerides, between about 30%-50% of the triacylglycerides present in the milk product can be substituted with palmitic acid (C16:0) at the sn-2 position on the glycerol backbone of the triacylglyceride. In some embodiments, the milk product comprises omega-6 and omega-3 fatty acids, the abundance of which can be represented as a ratio of omega-6 to omega-3, e.g., 10:1, 5:1, and the like. In some embodiments, the omega-6 to omega-3 fatty acid ratio ranges from about 0.75:1 to 10:1 or about 1.5 to 4.5. In some embodiments, the cultured milk product can include between about 100-160 g/L macromolecular content.

Having described the present disclosure, the same will be explained in greater detail in the following examples, which are included herein for illustration purposes only, and which are not intended to be limiting to the disclosure.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Production of Cultured Milk Components from Bovine Mammary Epithelium

In this example, bovine mammary epithelium is recapitulated and cultured milk product is produced in vitro. The system and process described is exemplary and can be scaled to produce multiliter volumes of milk components. Bovine mammary epithelial cells are expected to form a polarized monolayer on bioreactor fibers that have been precoated with one or more of laminin and collagen or other extracellular matrix proteins, as well as on uncoated fibers. When confluent, the monolayer forms a barrier that divides the intra- and extracapillary space (ECS), with the basal surface attached to the fibers and the apical surface oriented toward the ECS. Cultured milk component production is stimulated by addition of prolactin to the media. The secreted milk components are collected from the ECS and submitted for downstream analyses of the protein, lipid, and carbohydrate content in comparison to bovine milk produced in vivo.

Materials for use in this Example are shown in Table 3.

TABLE 3
Materials

| | | Catalog (or other |
|---|---|---|
| | | Identification) |
| Item | Supplier | Number |
| Primary bovine mammary | Academic community | MAC-T ¹ |
| epithelial cells | resource | |
| Dulbecco's Modified Eagle's | Sigma | D5030 |
| Medium (DMEM) | | |
| Fetal bovine serum | Sigma | F4135 |
| Insulin | Sigma | I6634 |
| Hydrocortisone | Sigma | H0888 |
| Dulbecco's phosphate buffered | ATCC | ATCC 30-2200 |
| saline (D-PBS) | | |
| Trypsin-EDTA | ATCC | PCS-999-003 |
| Trypsin Neutralizing Solution | ATCC | PCS-999-004 |
| EHS (Laminin-1/111) | Sigma | L2020-1 MG |
| Collagen-IV | Sigma | C5533-5 MG |
| Prolactin | Shenandoah | 100-45-500 ug |
| | Biotechnology | |

¹ Huynh, HT, et al., “Establishment of bovine mammary epithelial cells (MAC-T): an in vitro model for bovine lactation”, Exp Cell Res. 1991 Dec; 197(2): 191-9.

Procedures

Expansion of Primary Bovine Mammary Epithelial Cells (BMECs)

Bovine mammary epithelial cells (1 ampoule; 5×10⁵ cells) are expanded into one collagen-IV-coated T300 flask (or two T175 flasks) in DMEM supplemented with fetal bovine serum, insulin, and hydrocortisone, as listed in Table 3. Once an appropriate cell number is obtained, cells are rinsed with D-PBS and collected from the plates using trypsin-EDTA. Once cells are detached, trypsin activity is halted using Trypsin Neutralizing Solution. Cells are resuspended in medium and seeded into a hollow fiber bioreactor (Fibercell Systems), prepared as described below.

Preparation of Hollow Fiber Bioreactor (C2025D, 20 kD MWCO)

Prior to seeding, a bioreactor cartridge (Fibercell Systems) is prepared by pre-culturing with PBS for a minimum of 24 hours. The bioreactor cartridge is optionally pre-coated by adding about 50-100 μg of one or more of collagen I, collagen IV, laminin-111 (e.g., laminin-111 isolated from Engelbreth-Holm Swarm tumor), alpha-4, alpha-5, fibronectin, and/or entactin in 3.2 mL of PBS and allowing ultrafiltration across the fiber at room temperature overnight. The uncoated or precoated cartridge is exchanged with medium and incubated overnight at room temperature. The medium is then exchanged with the cells collected from the T300 (or T175) flask(s). The reservoir volume is no more than 125 mL. The cartridge is rotated 180 degrees after seeding the cells.

Cell Growth in the Bioreactor and Prolactin Stimulation

After seeding the bioreactor, cells are grown in DMEM supplemented with fetal bovine serum, insulin, and hydrocortisone.

Before stimulation of milk secretion, the medium in the ECS is flushed and replaced with PBS. To stimulate milk component secretion, lactogenic medium (medium supplemented with 5 μg/mL prolactin) is added. The lactogenic medium can also be supplemented with an elevated concentration of glucose and the essential dietary precursors for milk fatty acids, linoleic acid and α-linolenic acid. The bioreactor is maintained for 10 days with sampling as described below.

Harvesting and Sample Preparation

Samples, comprised of supernatant from the ECS and an equivalent volume of media from the reservoir, are collected once daily for 10 days after addition of prolactin to the media. The samples are spun in a centrifuge to collect any debris and resuspended in an equivalent volume of PBS. The supernatants from the ECS and media samples are divided into 0.5 mL aliquots in microfuge tubes and frozen at −80° C. The pellet debris is resuspended in a volume of PBS equivalent to the original sample and frozen at −80° C. Samples are processed to determine relative concentrations of milk components produced.

Scaling for Multiliter Production

To scale for multiliter production, the preceding procedure with relative adjustments in reagent volumes for a larger bioreactor (e.g., Fibercell Systems cat. no. C2018) is performed.

The foregoing examples are illustrative of the present disclosure and are not to be construed as limiting thereof. Although the disclosure has been described in detail with reference to preferred embodiments, variations and modifications exist within the scope and spirit of the disclosure as described and defined in the following claims.

What is claimed is:

1. A cultured milk product comprising:
about 28-40 grams per liter (g/L) protein components;
about 35-55 g/L lipid components;
about 0.01-0.15 g/L bovine milk oligosaccharides (BMOs); and
about 40-60 g/L lactose,
wherein at least one of the protein components, lipid components, MOs, and lactose is produced by cultured bovine mammary epithelial cells.

2. The cultured milk product according to claim 1, wherein the protein component comprises whey protein.

3. The cultured milk product according to claim 2, wherein the whey protein has a concentration of about 1-24 g/L in the milk product.

4. The cultured milk product according to either of claims 2 or 3, wherein the protein component further comprises casein protein.

5. The cultured milk product according to claim 1, wherein the protein component comprises casein protein.

6. The cultured milk product according to any one of claims 1, 4 or 5, wherein the protein component comprises beta-casein, kappa-casein and alpha-casein.

7. The cultured milk product according to claim 6, wherein the beta-casein has a concentration of about 7-12 g/L, the kappa-casein has a concentration of about 1-4 g/L, and the alpha-casein has a concentration of about 9-16 g/L in the milk product.

8. The cultured milk product according to any one of claims 5-7, wherein the alpha-casein comprises one or more of alphaₛ₁-casein and alphaₛ₂-casein.

9. The cultured milk product according to claim 8, wherein the alphaₛ₁-casein is more than about 3-fold more abundant than alphaₛ₂-casein.

10. The cultured milk product according to claim 9, wherein the alphaₛ₁-casein has a concentration of about 7-12 g/L in the milk product.

11. The cultured milk product according to either of claims 9 or 10, wherein the alphaₛ₂-casein has a concentration of about 2-4 g/L in the milk product.

12. The cultured milk product according to any one of claims 6-11, wherein the beta-casein comprises greater than about 50% of total casein content.

13. The cultured milk product according to any one of claims 2-12, wherein the protein component further comprises one or more of beta-lactoglobulin, alpha-lactalbumin, lysozyme, lactoferrin and serum albumin.

14. The cultured milk product according to claim 13, wherein the beta-lactoglobulin has a concentration of about 2-5 g/L in the milk product.

15. The cultured milk product according to claim 13 or 14, wherein the alpha-lactalbumin has a concentration of about 0.5-2 g/L in the milk product.

16. The cultured milk product according to any one of claims 13-15, wherein the lysozyme has a concentration of about 5-15 μg/L in the milk product.

17. The cultured milk product according to any one of claims 13-16, wherein the lactoferrin has a concentration of about 0.01-0.5 g/L in the milk product.

18. The cultured milk product according to any one of claims 13-17, wherein the serum albumin has a concentration of about 0.05-2 g/L in the milk product.

19. The cultured milk product according to any one of claims 1-18, wherein the protein components are of bovine origin.

20. The cultured milk product according to any of claims 1-19, wherein the lipid component comprises one or more of triacylglycerides, diacylglycerides, saturated fatty acids, monounsaturated fatty acids, polyunsaturated fatty acids, cholesterol, and phospholipids.

21. The cultured milk product according to claim 20, wherein the triacylglycerides have a concentration of about 30-54 g/L in the milk product.

22. The cultured milk product according to either claim 20 or 21, wherein the diacylglycerides have a concentration of about 0.33-2 g/L in the milk product.

23. The cultured milk product according to any one of claims 20-22, wherein the saturated fatty acids have a concentration of about 15-25 g/L in the milk product.

24. The cultured milk product according to any one of claims 20-23, wherein the saturated fatty acids comprise one or more of myristic acid, palmitic acid, and lauric acid.

25. The cultured milk product according to claim 24, wherein myristic acid has a concentration of about 1-4 g/L in the milk product.

26. The cultured milk product according to either of claims 24 or 25, wherein palmitic acid has a concentration of about 6-10 g/L in the milk product.

27. The cultured milk product according to any one of claims 24-26, wherein lauric acid has a concentration of about 0.6-1 g/L in the milk product.

28. The cultured milk product according to any one of claims 20-27, wherein monounsaturated fatty acids have a concentration of about 5-12 g/L in the milk product.

29. The cultured milk product according to claim 28, wherein the monounsaturated fatty acid comprises oleic acid.

30. The cultured milk product according to claim 29, wherein the oleic acid has a concentration of about 6-10 g/L in the milk product.

31. The cultured milk product according to any one of claims 20-30, wherein polyunsaturated fats have a concentration of about 0.5-10 g/L in the milk product.

32. The cultured milk product according to claim 31, wherein the polyunsaturated fats comprise one or more of linoleic acid, conjugated linoleic acid, and alpha-linoleic acid.

33. The cultured milk product according to claim 32, wherein linoleic acid has a concentration of about 0.5-2 g/L in the milk product.

34. The cultured milk product according to either of claims 32 or 33, wherein conjugated linolenic acid has a concentration of about 0.05-0.15 g/L in the milk product.

35. The cultured milk product according to any one of claims 32-34, wherein alpha-linoleic acid has a concentration of about 0.5-1.5 g/L in the milk product.

36. The cultured milk product according to either of claims 31 or 32, wherein linoleic acid has a concentration of about 0.5-2 g/L, conjugated linolenic acid has a concentration of about 0.05-0.15 g/L, and alpha-linoleic acid has a concentration of about 0.5-1.5 g/L in the milk product.

37. The cultured milk product according to any one of claims 20-36, wherein cholesterol has a concentration of about 0.2-4 g/L in milk product.

38. The cultured milk product according to any one of claims 20-37, wherein phospholipids have a concentration of about 0.1-1 g/L in the milk product.

39. The cultured milk product according to any one of claims 1-38, wherein the milk oligosaccharide component comprises one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL).

40. The cultured milk product according to claim 39, wherein the one or more milk oligosaccharides comprises 6′-Sialyllactose (6′-SL), which oligosaccharide has a concentration of about 0.01-0.1 g/L in the milk product.

41. The cultured milk product according to either claim 39 or 40, wherein the one or more oligosaccharides comprises 6′-sialyl-n-acetyllactosamine (6′-SLN), which oligosaccharide has a concentration of about 0.005-0.02 g/L in the milk product.

42. The cultured milk product according to any one of claims 39-41, wherein the one or more oligosaccharides comprises Disialyllactose (DSL), which oligosaccharide has a concentration of less than about 0.01 g/L in the milk product.

43. The cultured milk product according to any one of claims 39-42, wherein the one or more oligosaccharides comprises Galactosaminuyllactose (GNL), which oligosaccharide has a concentration of about 0.002-0.006 g/L in the milk product.

44. The cultured milk product according to any one of claims 39-43, wherein the one or more oligosaccharides comprises 3′-Sialyllactose (3′-SL), which oligosaccharide has a concentration of about 0.025-0.15 g/L in the milk product.

45. The cultured milk product according to any one of claims 39-44, wherein the milk product comprises about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL), and about 0.025-0.15 g/L 3′-SL (3′-sialyllactose).

46. A cultured milk product comprising:
about 28-40 grams per liter (g/L) protein components;
about 35-55 g/L lipid components;
about 0.01-0.15 g/L milk oligosaccharides (MOs); and
about 40-60 g/L lactose,
wherein the protein components comprise whey, beta-casein, kappa-casein, alphaₛ₁-casein, alphaₛ₂-casein, beta-lactoglobulin, alpha-lactalbumin, lysozyme, lactoferrin, and serum albumin,
wherein the lipid components comprise triacylglycerides, diacylglycerides, saturated fatty acids, monounsaturated fatty acids, polyunsaturated fatty acids, cholesterol, and phospholipids,
wherein the bovine milk oligosaccharides comprise 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL),
wherein saturated fatty acids comprise myristic acid, palmitic acid, and lauric acid,
wherein monounsaturated fatty acids comprises oleic acid,
wherein polyunsaturated fatty acids comprise linoleic acid, conjugated linoleic acid, and alpha-linoleic acid, and
wherein at least one of the protein components, lipid components, BMOs, and lactose is produced by cultured bovine mammary epithelial cells.

47. The cultured milk product according to claim 46, wherein the milk product comprises about 2-16 g/L whey, 7-12 g/L beta-casein, about 1-4 g/L kappa-casein, about 7-12 g/L alphaₛ₁-casein, about 2-4 g/L alphaₛ₂-casein, about 2-5 g/L beta-lactoglobulin, about 0.5-2 g/L alpha-lactalbumin, about 5-15 μg/L lysozyme, about 0.01-0.5 g/L lactoferrin, about 0.05-2 g/L serum albumin, about 30-54 g/L triacylglycerides, about 0.3-2 g/L diacylglycerides, about 15-25 g/L saturated fatty acids, about 5-12 g/L monounsaturated fatty acids, about 0.5-10 g/L polyunsaturated fatty acids, about 0.2-4 g/L cholesterol, about 0.1-1 g/L phospholipids, about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL) and about 0.025-0.15 g/L 3′-Sialyllactose (3′-SL),
wherein the saturated fatty acids comprise about 1-4 g/L myristic acid, about 6-10 g/L palmitic acid, and about 0.6-1 g/L lauric acid,
wherein the monounsaturated fatty acids comprises about 6-10 g/L oleic acid, and
wherein the polyunsaturated fatty acids comprise about 0.5-2 g/L linoleic acid, about 0.05-0.15 g/L conjugated linoleic acid, and about 0.5-1.5 g/L alpha-linoleic acid.

48. The cultured milk product according to any of claims 1-47, wherein the protein components, lipid components, BMOs, and lactose are isolated from cultured bovine mammary epithelial cells.

49. The cultured milk product according to any of claims 1-48, wherein the cultured bovine mammary epithelial cells comprise one or more immortalized bovine mammary cell lines.

50. The cultured milk product according to any of claims 1-48, wherein the cultured bovine mammary epithelial cells are derived from one or more primary bovine mammary tissue samples.

51. The cultured milk product according to claim 50, wherein the one or more primary bovine mammary tissue samples is derived from a surgical explant of bovine mammary gland tissue.

52. The cultured milk product according to either claim 50 or 51, wherein the one or more primary bovine mammary tissue samples comprises tissue or cells collected from bovine mammary parenchyma.

53. The cultured milk product according to either claim 50 or 52, wherein the one or more primary bovine mammary tissue samples is derived from a needle aspiration of bovine mammary gland tissue.

54. The cultured milk product according to any one of claims 50-53, wherein the primary bovine mammary tissue further comprises one or more myoepithelial cells.

55. The cultured milk product according to any of claims 50-54, wherein the primary bovine mammary tissue further comprises one or more stem cells.

56. The cultured milk product according to claim 48, wherein the cultured bovine mammary epithelial cells are co-cultured with one or more myoepithelial cell lines.

57. The cultured milk product according to either of claims 48 or 56, wherein the cultured bovine mammary epithelial cells are co-cultured with one or more stem cell lines.

58. A cultured milk product according to any one of claims 1-57 that is sterile.

59. A cultured milk product according to claim 58 that is sterile without pasteurization.

60. A cultured milk product according to either of claims 58 or 59 that is free of immunoglobulin protein.

61. The cultured milk product according to any of claims 1-60, wherein the cultured milk product comprises at least about 80%, or at least about 85%, or at least about 90%, or at least about 95%, or at least about 97%, or at least about 98%, or at least about 99%, of the overall macromolecular composition of bovine milk.

62. The cultured milk product according to any of claims 1-61, wherein non-protein nitrogen content comprises at least about 10%, or at least about 15%, or at least about 20%, or at least about 25%, or at least about 30% of total nitrogen content.

63. A frozen cultured milk product, comprising the cultured milk product according to any one of claims 1-62, 89 and 90 that has been frozen.

64. A lyophilized cultured milk product, comprising the cultured milk product according to any one of claims 1-62, 89 and 90 that has been lyophilized.

65. A containerized cultured milk product, comprising the cultured milk product, according to any one of claims 1-62, 89 and 90 that is packaged into a container.

66. A containerized frozen cultured milk product, comprising the frozen cultured milk product according to claim 65 that is packaged into a container.

67. A containerized lyophilized cultured milk product, comprising the lyophilized cultured milk product according to claim 64 that is packaged into a container.

68. An extracted cultured milk product, comprising one or more components extracted from the cultured milk product according to any one of claims 1-64, 89 and 90.

69. The extracted cultured milk product according to claim 68, wherein the one or more components extracted from the collected milk product are lyophilized or concentrated to produce a lyophilized or a concentrated extracted cultured milk product component.

70. The extracted cultured milk product according to claim 69, wherein the one or more components extracted from the collected cultured milk product are concentrated by membrane filtration or reverse osmosis.

71. The extracted cultured milk product according to any one of claims 68-70, wherein the one or more extracted components from the collected cultured milk product comprise milk protein, lipid, carbohydrate, vitamin, and minerals.

72. A containerized extracted cultured milk product, comprising the extracted cultured milk product according to any one of claims 68-71 that is packaged in a container.

73. The containerized cultured milk product according to any of claims 65-67 or 72, wherein the container is sterile.

74. The containerized cultured milk product according to any of claims 65-67, 72, or 73, wherein the container is vacuum-sealed

75. The containerized cultured milk product according to any one of claims 65-67 or 72-74, wherein the container is a food grade container.

76. The containerized cultured milk product according to any one of claims 65-67 or 72-75, wherein the container is a canister, a jar, a bottle, a bag, a box, or a pouch.

77. The cultured milk product according to any one of claims 1-62, 89, and 90 comprising about 450-900 kcal/L available energy content.

78. The cultured milk product according to claim 77, wherein between about 40-60% of the available energy content is from lipids.

79. The cultured milk product according to any one of claims 1-62, 77, 78, 89, and 90, wherein the milk product comprises between about 100 and 160 g/L macromolecular content.

80. The cultured milk product according to any one of claims 4-12, wherein the casein protein is complexed into one or more micelle structures with a diameter between about 100 nm and 200 nm.

81. The cultured milk product according to any one of claims 1-62, 77-80, 89, and 90, comprising between about 10⁹ to 10¹¹ milk fat globules per milliliter, wherein the milk fat globules have a core comprising one or more triacylglycerides, the core surrounded by a trilayer comprising one or more phospholipids and one or more membrane proteins.

82. The cultured milk product according to any one of claims 20-21, 46-47, 81, 89, and 90, wherein between about 30%-50% of the triacylglycerides present in the cultured milk product are substituted with palmitic acid (C16:0) at a sn-2 position.

83. The cultured milk product according to any one of claims 1-62, 77-82, 89, and 90, comprising an omega-6 to omega-3 fatty acid ratio of about 1.5 to 4.5.

84. The cultured milk product according to any one of claims 1-62, 77-83, 89, and 90, wherein the cultured milk product comprises between about 100-160 g/L macromolecular content.

85. A filtered cultured milk product, comprising one or more components filtered from the cultured milk product according to any one of claims 1-64 and 89.

86. The filtered cultured milk product according to claim 85, wherein the one or more components filtered from the collected milk product are lyophilized or concentrated to produce a lyophilized or a concentrated filtered cultured milk product component.

87. The filtered cultured milk product according to claim 86, wherein the one or more components filtered from the collected cultured milk product are concentrated by membrane filtration or reverse osmosis.

88. The filtered cultured milk product according to any one of claims 85-87, wherein the one or more filtered components from the collected cultured milk product comprise milk protein, lipid, carbohydrate, vitamin, and minerals.

89. The cultured milk product according to any of claims 48-64, wherein the protein components, lipid components, BMOs, and lactose are isolated from a secretion that is produced by the cultured bovine mammary epithelial cells.

90. The cultured milk product according to any of claims 48-64, wherein the protein components, lipid components, BMOs, and lactose are isolated from a supernatant from the cultured bovine mammary epithelial cells.

91. The cultured milk product according to any of claims 1-48, wherein the cultured bovine mammary epithelial cells are derived from one or more bovine mammary epithelial cells isolated from raw bovine milk.

92. The cultured milk product according to any of claims 1-60, wherein the antioxidant capacity of the cultured milk products is substantially different from the antioxidant capacity of bovine milk.

93. The cultured milk product according to any of claims 1-60, wherein the cultured milk product does not comprise or is substantially free of one or more contaminants and toxins of bovine milk.

94. The cultured milk product according to any of claims 1-60, wherein the cultured milk product does not comprise or is substantially free of one or more contaminants of bovine milk selected from the group comprising hormones (e.g., pituitary, steroid, hypothalamic, and thyroid hormones), gastrointestinal peptides (e.g., nerve and epidermal growth factors, and the growth inhibitors MDGI and MAF), rBGH or recombinant bovine growth hormone (a genetically engineered hormone injected into cows to increase milk production), pus from infected bovine udders, and/or antibiotics or pharmaceuticals which have been administered to cows.

95. The cultured milk product according to any of claims 1-60, wherein the cultured milk product does not comprise or is substantially free of one or more pathogens or microorganisms of bovine milk.

96. The cultured milk product according to any of claims 1-60, wherein the cultured milk product does not comprise or is substantially free of one or more pathogens or microorganisms of bovine milk selected from the group comprising Brucella, Campylobacter jejuni, Coliforms, Coxiella burnetii, Escherichia coli, Listeria monocytogenes, Mycobacterium bovis and tuberculosis, Mycobacterium paratuberculosis, Psychrotrophic Bacteria, Salmonella spp., Yersinia enterocolitica, Proteobacteria, Bacteroidetes, Firmicutes, Actinobacteria, Acidobacteria, Pseudomonas, Brevibacteriaceae, Corynebacteriaceae, Staphylococcaceae, Arthrobacter, Cronobacter, Ruminococcus and/or Faecalibacterium.
